(19) United States
(12) Patent Application Publication
(10) Pub. No.: US 2010/0298939 A1
(43) Pub. Date: Nov. 25, 2010

(54) Intervertebral Disc Implant

(75) Inventors: Daniel Delfosse, Jegensdorf (CH); Dirk Salomon, Jonaswalde (DE)

Correspondence Address: SCULLY SCOTT MURPHY & PRESSER, PC, 400 GARDEN CITY PLAZA, SUITE 300, GARDEN CITY, NY, 11530, US

(73) Assignee: Mathys AG Bettlach, Bettlach (CH)

(21) Appl. No.: 11/921,479

(22) PCT Filed: Mar. 22, 2006
(86) PCT No.: PCT/EP2006/002640

§ 371 Date: Dec. 28, 2007

(30) Foreign Application Priority Data
Jun. 3, 2005 (DE) 102005025685.6

(51) Int. Cl. A61F 2/44

(52) U.S. Cl. 623/17.16

(57) ABSTRACT

The invention relates to an intervertebral disk implant (1) which is used to implant between two vertebrae (2), comprising at least two components (3,4; 13,14,15) which form at least one sliding pair and which are made of a ceramic material. The at least two ceramic components (3,4; 13,14,15) are arranged in a covering (5).

The invention relates to a mobile intervertebral disc implant, in particular an implant with a ceramic/ceramic joint.

An artificial intervertebral disc is known, for example, from EP 1 287 794 A1, which comprises two end plates and a resiliently deformable disc braced axially under prestress between the end plates. The disc is located inside a tubular, resilient fibre ring, the end plates being in tension-resistant connection with the fibre ring. The end plates are made from metal.

A particular disadvantage of the artificial intervertebral disc known from EP 1 287 794 A1 is its poor sliding behaviour. Although the intervertebral disc according to the prior art is readily able to absorb axial expansion and compression, the intervertebral disc may react badly in the event of displacement between the vertebrae or in particular in the event of inclination of the vertebrae towards one another.

This may be circumvented by using ceramic implants, which have better sliding behaviour. However, these have the serious disadvantage that, in the event of breakage of the relatively brittle ceramic material, the risk of injury to the patient equipped with the prosthesis is very high and may have serious consequences, even to the extent of paraplegia or death, if splinters of the ceramic implant enter the spinal cord canal.

The object of the invention is therefore to provide an intervertebral disc implant which on the one hand exhibits good sliding behaviour and on the other hand is so designed that damage to the implant cannot threaten the health of the patient.

The object is achieved with the features of claim 1.

According to the invention, the ceramic intervertebral disc implant is encased in a casing which on the one hand is strong enough to retain the fragments of the implant in the event of damage and on the other hand is sufficiently flexible to allow the natural, unlimited mobility of the implant which is physiologically necessary.

Further advantageous developments of the invention are indicated in the subclaims.

Exemplary embodiments of the invention are explained in greater detail below with reference to partially schematic drawings, in which:

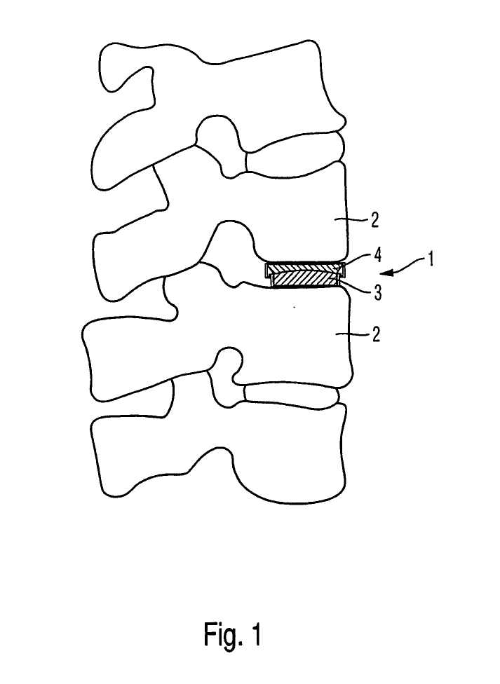

FIG. 1 is a schematic view of a first exemplary embodiment of an intervertebral implant according to the invention, in situ,

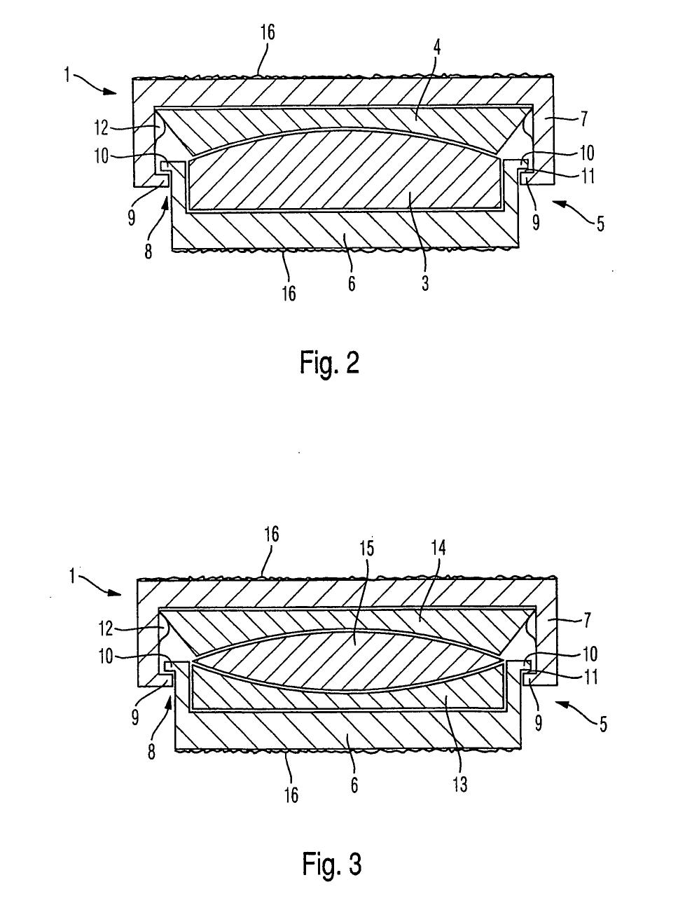

FIG. 2 is an enlarged sectional representation in accordance with FIG. 1 of the first exemplary embodiment of the intervertebral disc implant according to the invention, and

FIG. 3 shows a second exemplary embodiment of an intervertebral disc implant according to the invention in the same view as FIG. 2.

FIG. 1 is a highly schematic lateral representation of an intervertebral disc implant 1 according to the invention between two vertebrae 2 of a human spinal column.

The intervertebral disc implant 1 has to ensure a physiological movement process, with tilting towards one another of the two components 3 and 4 forming the intervertebral disc implant in the first exemplary embodiment being of particular importance. In this respect, the way components 3 and 4 slide on one another plays a decisive role. In contrast with the previously conventional plastics implants which replace defective intervertebral discs, a sliding pair comprising two components 3 and 4 consisting of ceramic material displays better sliding behaviour. In particular, static friction is less even under heavy loads.

However, the problem with ceramic intervertebral disc implants 1 is the high risk of injury resulting in serious injury to the extent of paraplegia or death if, in the event of breakage of the ceramic intervertebral disc implants 1, individual fragments reach the spinal cord canal and penetrate therein with their sharp broken edges.

To prevent this, it is proposed according to the invention to provide the two ceramic components 3 and 4 with a casing 5, which is made from a plastics material, preferably a biocompatible polymer such as polyethylene (PE), polyetheretherketone (PEEK), polyetherketoneketone (PEKK), polyetheracrylketone (PEAK), polyacryletherketone (PAEK) or polymethyl methacrylate (PMMA), and encloses the ceramic intervertebral disc implant 1 on all sides.

Preferably, the casing 5 is in two parts, so as to ensure easy assembly prior to implantation. The two parts 6 and 7 of the casing comprise an area 8 of overlap, in which two suitably shaped collar-like projections 9 and 10 engage one under the other such that, one the one hand, a movement gap 11 remains, which permits to the full extent the desired movement radius of approx. 10° and, on the other hand, the ceramic components 3 and 4 are reliably enclosed.

A circumferential ring 12 may be provided in the casing 5, in particular in the part 7 which is radially larger, said ring enabling stabilisation of the component 4 in the part 7 and being suitable as a fixing means in the event of possible preassembly. The circumferential ring 12 may take the form of an inwardly extending bead in one piece with the part 7 or may be subsequently inserted and fixed in place.

FIG. 3 is a schematic sectional representation, in the same view as FIG. 2, of a second exemplary embodiment of an intervertebral disc implant 1 according to the invention. Identical components are provided with matching reference numerals.

The second exemplary embodiment illustrated in FIG. 3 of an intervertebral disc implant 1 according to the invention is provided with three ceramic components 13, 14, 15, the components 13 and 14 corresponding to the components designated 3 and 4 in FIG. 2. Between the components 13 and 14 there is provided a third, likewise ceramic, lens-shaped component 15, which is embedded in the two components 13 and 14, which are of concave construction in the second exemplary embodiment. An intervertebral disc implant 1 constructed in this way provides great mobility while also being of small structural height. The casing 5 is constructed as in the first exemplary embodiment illustrated in FIG. 2.

A further advantage of a casing 5 according to the invention is the small structural height of only 4 mm to 8 mm. This is important, since ceramic intervertebral disc implants 1 have overall a small structural height which cannot be exceeded due to fitting conditions.

To minimise component dimensions, fibre reinforced parts 6, 7, for example of carbon fibre reinforced PEEK, may therefore also be used.

On its faces facing the vertebrae 2 the casing 5 may be provided with a titanium coating 16. This leads to roughening of the faces and improved osseo-integration.

The invention is not limited to the exemplary embodiments illustrated and is also suitable for further embodiments of intervertebral disc implants 1. All features of the invention may be combined in any desired manner.

1. An intervertebral disc implant (1) for implanting between two vertebrae (2), comprising at least two components (3, 4; 13, 14, 15), which form at least one sliding pair and which are made from a ceramic material, wherein the at least two ceramic components (3, 4; 13, 14, 15) are arranged in a casing (5).

2. An intervertebral disc implant according to claim 1,
characterised in that
the casing (5) consists of two parts (6, 7).

3. An intervertebral disc implant according to claim 2,
characterised in that
the parts (6, 7) of the casing (5) may be connected detachably together.

4. An intervertebral disc implant according to claim 2 or claim 3,
characterised in that
the parts (6, 7) of the casing (5) each comprise a circumferential collar-like projection (9, 10).

5. An intervertebral disc implant according to claim 4,
characterised in that
the collar-like projections (9, 10) engage one under the other, forming a movement gap (11).

6. An intervertebral disc implant according to one of claims 2 to 5,
characterised in that
a circumferential ring (12) is formed in one or both of the parts (6, 7) of the casing (5).

7. An intervertebral disc implant according to claim 6,
characterised in that
the component (3, 4; 13, 14) of the intervertebral disc implant (1) enclosed in the respective part (6, 7) of the casing (5) is fixed in place by the ring (12).

8. An intervertebral disc implant according to claim 6 or claim 7,
characterised in that
the ring (12) takes the form of a bead in one piece with the respective part (6, 7).

9. An intervertebral disc implant according to claim 6 or claim 7, characterised in that
the ring (12) takes the form of a separate component connectable to the respective part (6, 7).

10. An intervertebral disc implant according to one of claims 1 to 9,
characterised in that
the casing (5) consists of a biocompatible plastics material.

11. An intervertebral disc implant according to claim 10,
characterised in that
the plastics material is a polymer, in particular polyethylene (PE), polyetheretherketone (PEEK), polyetherketoneketone (PEKK), polyetheracrylketone (PEAK), polyacryletherketone (PAEK) or polymethyl methacrylate (PMMA).

12. An intervertebral disc implant according to one of claims 1 to 11,
characterised in that
the casing (5) is provided with a coating (16) on its faces facing the vertebrae (2).

13. An intervertebral disc implant according to claim 12,
characterised in that
the coating (16) consists of titanium.

14. An intervertebral disc implant according to one of claims 1 to 13,
characterised in that
the casing (5) of the intervertebral disc implant (1) has an axial height of 4 mm to 8 mm.

15. An intervertebral disc implant according to one of claims 1 to 14,
characterised in that
the casing (5) is fibre-reinforced.

16. An intervertebral disc implant according to claim 15,
characterised in that
the fibre reinforcement consists of carbon fibre.
